(19) United States
(12) Patent Application Publication
(10) Pub. No.: US 2026/0363743 A1
(43) Pub. Date: Nov. 19, 2026

(54) ANGLED BONE PLATES

(71) Applicant: RTG Scientific, LLC, Austin, TX (US)

(72) Inventor: Bruce HOFFMAN, Smithfield, UT (US)

(73) Assignee: RTG Scientific, LLC, Austin, TX (US)

(21) Appl. No.: 19/680,956

(22) Filed: May 18, 2026

Related U.S. Application Data
(60) Provisional application No. 63/807,723, filed on May 18, 2025.

(51) Int. Cl. A61B 17/80

(52) U.S. Cl. CPC A61B 17/80

(57) ABSTRACT

An angled bone plate may include a bone-facing surface, a reverse surface, a ridge, a first leg projecting away from the ridge at a first leg angle with a first longitudinal contact surface configured to contact the bone along a first longitudinal surface area on the bone, and a second leg projecting away from the ridge at a second leg angle with a second longitudinal contact surface configured to contact the bone along a second longitudinal surface area on the bone. A sum of the first leg angle and the second leg angle may comprise a total leg angle of the angled bone plate. An intermediate portion of the bone-facing surface between the first and second contact surfaces may be configured to project away from and disengage an intermediate surface on the bone positioned between the first and second contact surfaces.

### CROSS-REFERENCE TO RELATED APPLICATIONS

This application claims the benefit of U.S. Provisional Patent Application No. 63/807,723 filed on May 18, 2025, entitled ANGLED BONE PLATES, which is incorporated by reference as though set forth herein in its entirety.

### TECHNICAL FIELD

The present disclosure relates to bone plates, systems, and methods. More specifically, the present disclosure relates to improved angled bone plate designs, systems, and methods for stabilizing bone.

### BACKGROUND

Small bone plates for stabilizing a bone with fractures or defects are typically either flat in shape (e.g., see the example flat bone plate 16 shown in FIGS. 2 and 4), or curved in shape (e.g., see the example curved bone plate 15 shown in FIGS. 1 and 3).

Flat bone plates may not conform well to the curved surfaces of the bone. Curved bone plates may conform better to the curved surfaces of the bone, but the enlarged contact patches of curved bone plates can restrict blood flow to the periosteum of the bone, thereby reducing bone health and healing. Moreover, both flat and curved bone plates are more susceptible to bending and torsional/twisting forces due to their respective cross-sectional shapes (e.g., see FIGS. 3 and 4) and associated moments of inertia.

Accordingly, improved bone plate designs would be desirable that: (1) are stronger against bending and torsional forces; (2) allow for smaller bone plates with equivalent strength vs. larger flat/curved bone plates (e.g., a 2.7mm angled bone plate of the present disclosure can be comparable in strength to a typical 3.5mm flat/curved bone plate, etc.); (3) allow for a greater plate hole density with equivalent strength, vs. larger flat/curved bone plates, to provide more fastener fixation options in bone (e.g., a 2.7mm angled bone plate of the present disclosure can have a plate hole density of 0.14 holes/mm with equivalent strength to a typical 3.5mm flat/curved bone plate with a plate hole density of 0.08 holes/mm, etc.); (4) achieve better seating characteristics against the bone by “straddling” the bone rather than trying to precisely conform to the complex surface shape of the bone; (5) are easier and quicker to install due to “straddling” the bone vs. flat/curved bone plates; and (6) do not restrict blood flow to the periosteum by engaging the bone via a minimal contact patch to encourage bone health and healing.

### SUMMARY

The bone plates, systems, and methods of the present disclosure have been developed in response to the present state of the art, and in particular, in response to the problems and needs in the art that have not yet been fully solved by currently available bone plates, systems, and methods. In some embodiments, the bone plates, systems, and methods of the present disclosure may provide improved bone plates, systems, and methods for stabilizing bone. It will be understood that the below summary is illustrative only and should not be regarded as limiting.

In some embodiments, an angled bone plate for stabilizing a bone may include: a bone-facing surface; a reverse surface opposite the bone-facing surface; a ridge extending along a longitudinal plate axis of the angled bone plate; a first leg projecting away from the ridge at a first leg angle with respect to a transverse plate axis of the angled bone plate, the first leg including a first longitudinal contact surface along the bone-facing surface of the first leg that may be configured to contact the bone along a first longitudinal surface area on the bone; and a second leg projecting away from the ridge at a second leg angle with respect to the transverse plate axis of the angled bone plate, the second leg including a second longitudinal contact surface along the bone-facing surface of the second leg that may be configured to contact the bone along a second longitudinal surface area on the bone. A sum of the first leg angle and the second leg angle may comprise a total leg angle of the angled bone plate that may be obtuse. An intermediate portion of the bone-facing surface of the angled bone plate between the first longitudinal contact surface and the second longitudinal contact surface of the angled bone plate may be configured to project away from and disengage an intermediate surface of the bone positioned between the first longitudinal surface area and the second longitudinal surface area on the bone.

The angled bone plate according to any preceding paragraph, wherein the first leg may include a first leg width, and the second leg may include a second leg width.

The angled bone plate according to any preceding paragraph, wherein the first leg width may be equal to the second leg width.

The angled bone plate according to any preceding paragraph, wherein the first leg width may be less than the second leg width.

The angled bone plate according to any preceding paragraph, wherein the first leg width may be greater than the second leg width.

The angled bone plate according to any preceding paragraph, wherein a height of the angled bone plate may be equal to a width of the angled bone plate.

The angled bone plate according to any preceding paragraph, wherein a height of the angled bone plate may be less than a width of the angled bone plate.

The angled bone plate according to any preceding paragraph, wherein a height of the angled bone plate may be greater than a width of the angled bone plate.

In some embodiments, an angled bone plate for stabilizing a bone may include: a bone-facing surface; a reverse surface opposite the bone-facing surface; a ridge extending along a longitudinal plate axis of the angled bone plate; a first leg projecting away from the ridge at a first leg angle with respect to a transverse plate axis of the angled bone plate, the first leg including a first longitudinal contact surface along the bone-facing surface of the first leg that may be configured to contact the bone along a first longitudinal surface area on the bone; and a second leg projecting away from the ridge at a second leg angle with respect to the transverse plate axis of the angled bone plate, the second leg including a second longitudinal contact surface along the bone-facing surface of the second leg that may be configured to contact the bone along a second longitudinal surface area on the bone. A sum of the first leg angle and the second leg angle may comprise a total leg angle of the angled bone plate that may be at least 90 degrees. An intermediate portion of the bone-facing surface of the angled bone plate between the first longitudinal contact surface and the second longitudinal contact surface of the angled bone plate may be configured to project away from and disengage an intermediate surface of the bone positioned between the first longitudinal surface area and the second longitudinal surface area on the bone.

The angled bone plate according to any preceding paragraph, wherein the first leg angle may be equal to the second leg angle.

The angled bone plate according to any preceding paragraph, wherein the first leg angle may be less than the second leg angle.

The angled bone plate according to any preceding paragraph, wherein the first leg angle may be greater than the second leg angle.

The angled bone plate according to any preceding paragraph, wherein the ridge may include a convex superior ridge surface extending along the longitudinal plate axis and a concave inferior ridge surface extending along the longitudinal plate axis opposite the convex superior ridge surface.

The angled bone plate according to any preceding paragraph, wherein the convex superior ridge surface may include at least one of a curved convex superior ridge surface shape and a pointed convex superior ridge surface shape, and the concave inferior ridge surface may include at least one of a curved concave inferior ridge surface shape and a pointed concave inferior ridge surface shape.

In some embodiments, an angled bone plate for stabilizing a bone may include: a bone-facing surface; a reverse surface opposite the bone-facing surface; a ridge extending along a longitudinal plate axis of the angled bone plate; a first leg projecting away from the ridge at a first leg angle with respect to a transverse plate axis of the angled bone plate, the first leg including a first longitudinal contact surface along the bone-facing surface of the first leg that may be configured to contact the bone along a first longitudinal surface area on the bone; and a second leg projecting away from the ridge at a second leg angle with respect to the transverse plate axis of the angled bone plate, the second leg including a second longitudinal contact surface along the bone-facing surface of the second leg that may be configured to contact the bone along a second longitudinal surface area on the bone. A sum of the first leg angle and the second leg angle may comprise a total leg angle of the angled bone plate that may be greater than 60 degrees. An intermediate portion of the bone-facing surface of the angled bone plate between the first longitudinal contact surface and the second longitudinal contact surface of the angled bone plate may be configured to project away from and disengage an intermediate surface of the bone positioned between the first longitudinal surface area and the second longitudinal surface area on the bone.

The angled bone plate according to any preceding paragraph, wherein the first longitudinal contact surface of the angled bone plate may include a first width, and the second longitudinal contact surface of the angled bone plate comprises a second width.

The angled bone plate according to any preceding paragraph, wherein the first width may be equal to the second width.

The angled bone plate according to any preceding paragraph, wherein the first width may be less than the second width.

The angled bone plate according to any preceding paragraph, wherein the first width may be greater than the second width.

The angled bone plate according to any preceding paragraph, wherein the intermediate portion of the bone-facing surface may include a third width that may be greater than a sum of the first width and the second width.

The angled bone plate according to any preceding paragraph, wherein the angled bone plate may further include at least one plate hole formed therethrough that may be configured to receive at least one fastener therein to couple the angled bone plate to a bone.

The angled bone plate according to any preceding paragraph, wherein the at least one plate hole may include at least one of: a fixed-angle locking hole, a variable angle locking hole, a non-locking hole, and a combination hole.

These and other features and advantages of the present disclosure will become more fully apparent from the following description and appended claims or may be learned by the practice of the bone plates, systems, and methods set forth hereinafter.

### BRIEF DESCRIPTION OF THE DRAWINGS

Exemplary embodiments of the present disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the present disclosure will be described with additional specificity and detail through use of the accompanying drawings in which:

FIG. 1 illustrates a perspective top view of a curved bone plate, according to an example of the present disclosure;

FIG. 2 illustrates a perspective top view of a flat bone plate, according to an example of the present disclosure;

FIG. 3 illustrates an end view of the curved bone plate of FIG. 1;

FIG. 4 illustrates an end view of the curved bone plate of FIG. 2;

FIG. 5A illustrates a perspective top view of an angled bone plate, according to an embodiment of the present disclosure;

FIG. 5B illustrates a perspective bottom view of the angled bone plate of FIG. 5A;

FIG. 5C illustrates a side view of the angled bone plate of FIG. 5A;

FIG. 5D illustrates a bottom view of the angled bone plate of FIG. 5A;

FIG. 5E illustrates a top view of the angled bone plate of FIG. 5A;

FIG. 5F illustrates a longitudinal cross-sectional side view of the angled bone plate taken along the line A-A in FIG. 5E;

FIG. 5G illustrates a close up view of one of the plate holes from FIG. 5F;

FIG. 5H illustrates a transverse cross-sectional side view of the angled bone plate taken along the line B-B in FIG. 5E;

FIG. 6 illustrates an end view of the angled bone plate of FIG. 5A placed on a bone;

FIG. 7A illustrates a perspective distal end view of a fastener, according to an embodiment of the present disclosure;

FIG. 7B illustrates a perspective proximal end view of the fastener of FIG. 7A;

FIG. 7C illustrates a distal end view of the fastener of FIG. 7A with a dual thread start configuration;

FIG. 7D illustrates a distal end view of the fastener of FIG. 7A with a triple thread start configuration;

FIG. 7E illustrates a side view of the fastener of FIG. 7A;

FIG. 7F illustrates a cross-sectional side view of the fastener taken along the line C-C in FIG. 7E;

FIG. 7G illustrates a close up view of the head of the fastener shown in FIG. 7F;

FIG. 8A illustrates a perspective distal end view of a fastener, according to another embodiment of the present disclosure;

FIG. 8B illustrates a perspective proximal end view of the fastener of FIG. 8A;

FIG. 8C illustrates a side view of the fastener of FIG. 8A;

FIG. 8D illustrates a cross-sectional side view of the fastener taken along the line D-D in FIG. 8C; and

FIG. 8E illustrates a close up view of the head of the fastener shown in FIG. 8D.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word "exemplary" is used herein to mean "serving as an example, instance, or illustration." Any embodiment described herein as "exemplary" is not necessarily to be construed as preferred or advantageous over other embodiments. While the various aspects of the embodiments are presented in the figures, the figures are not necessarily drawn to scale unless specifically indicated.

Standard medical planes of reference and descriptive terminology are employed in this specification. While these terms are commonly used to refer to the human body, certain terms may also be applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means outboard deviation of the knees (away from the sagittal plane) from the line between the hip and ankle, resulting in a “bowlegged” stance. Valgus means inboard deviation of the knees (toward the sagittal plane) from the line between the hip and ankle, resulting in a “knock-kneed” stance.

Although the present disclosure illustrates angled plates in the context of stabilizing bone, it will also be understood that the angled plates, fasteners, and methods disclosed herein may be adapted for use with any substrate or material type including, but not limited to: tissue(s) such as cartilage and other soft tissue(s), metal(s), metal alloy(s), plastic(s), ceramic(s), polymer(s), composite material(s), wood material(s), stone material(s), cement/concrete material(s), etc.

As used herein, the term “fastener” can comprise any fastener type that may be made of any type of material(s) and adapted to couple the angled bone plate embodiments described or contemplated herein to any type of substrate(s) or substrate material(s).

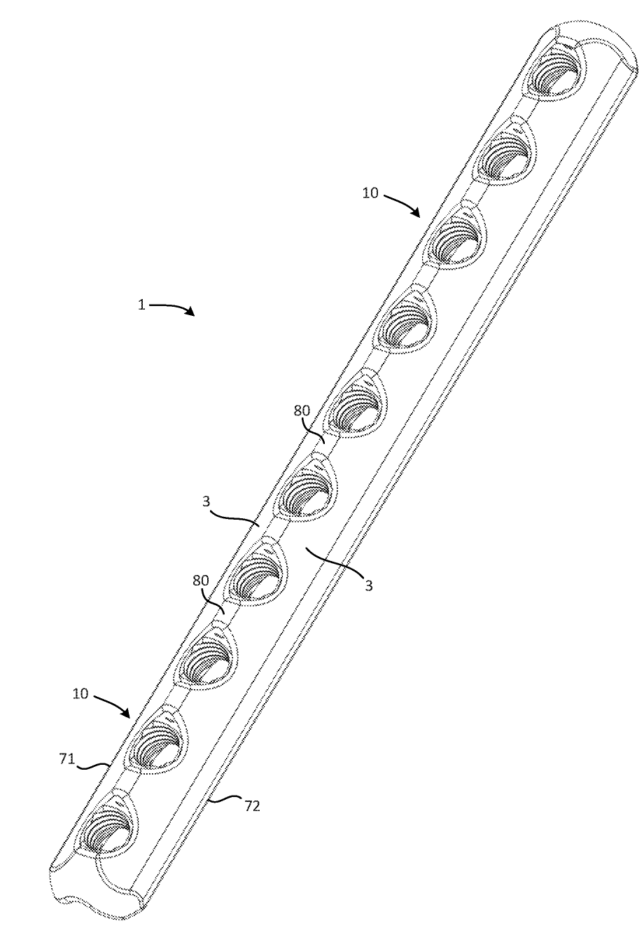

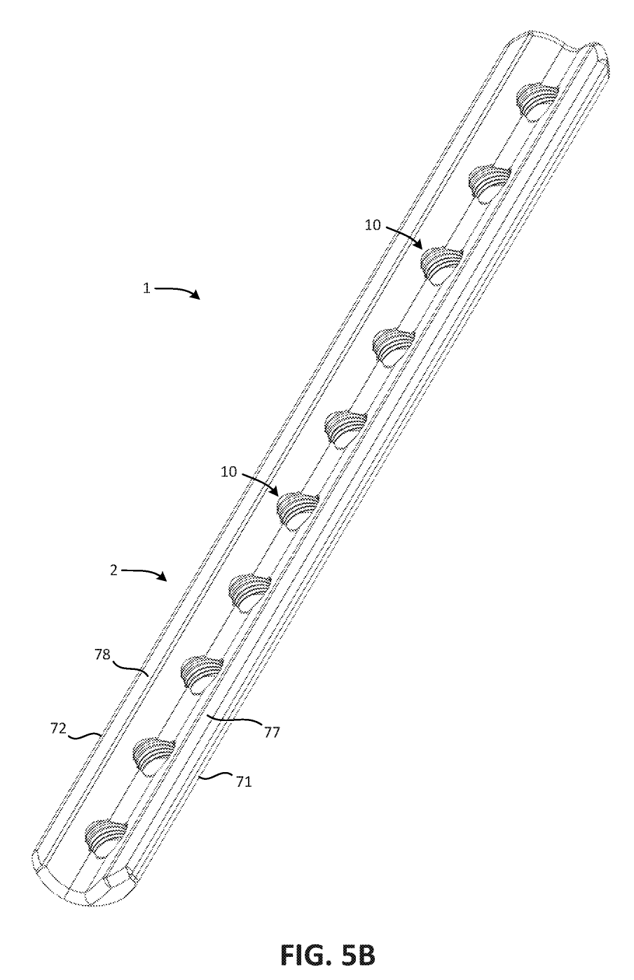

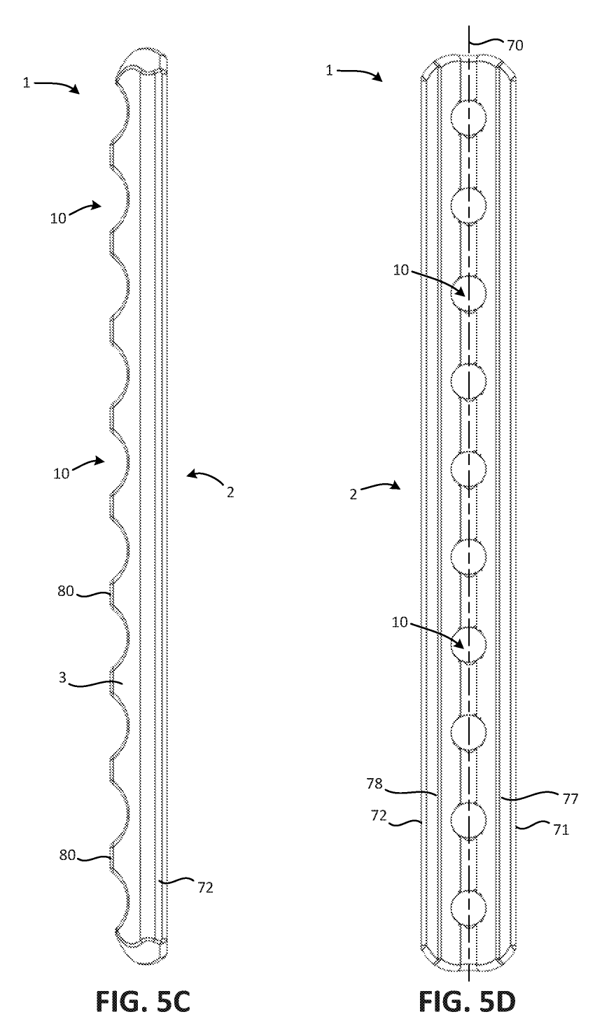

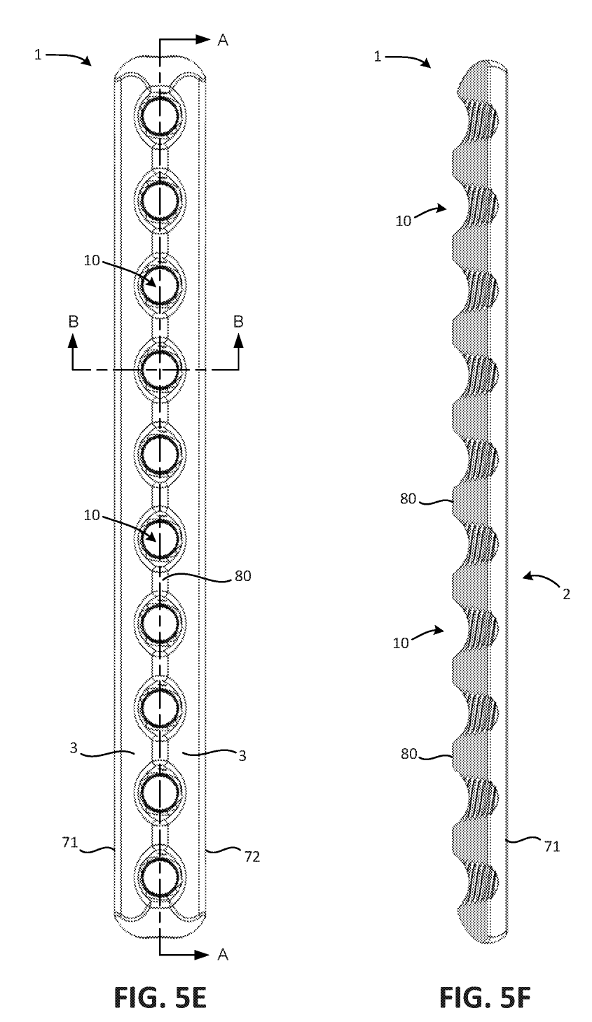

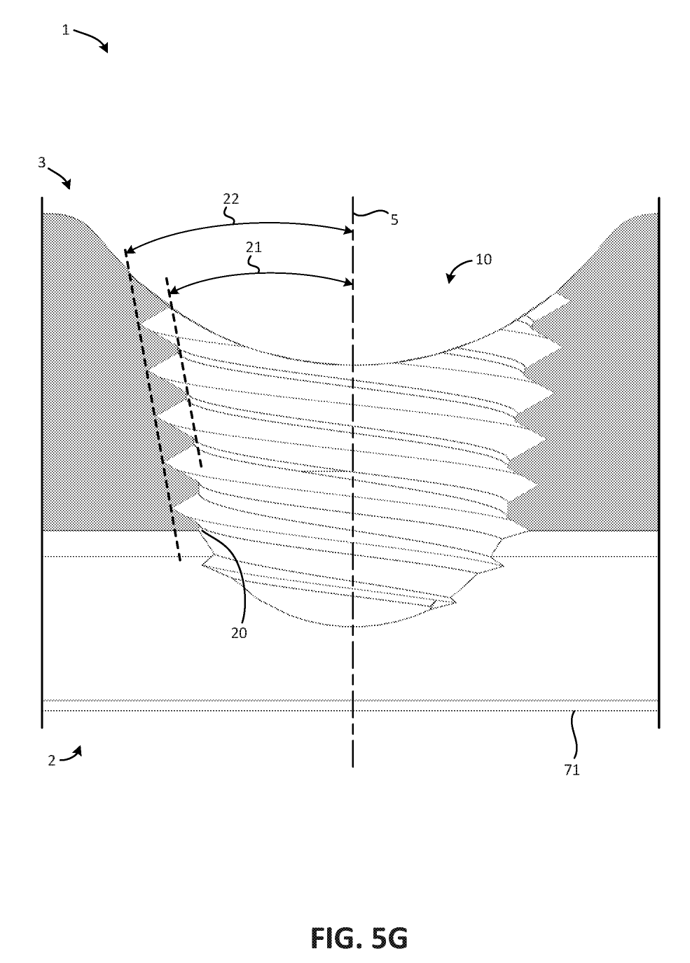

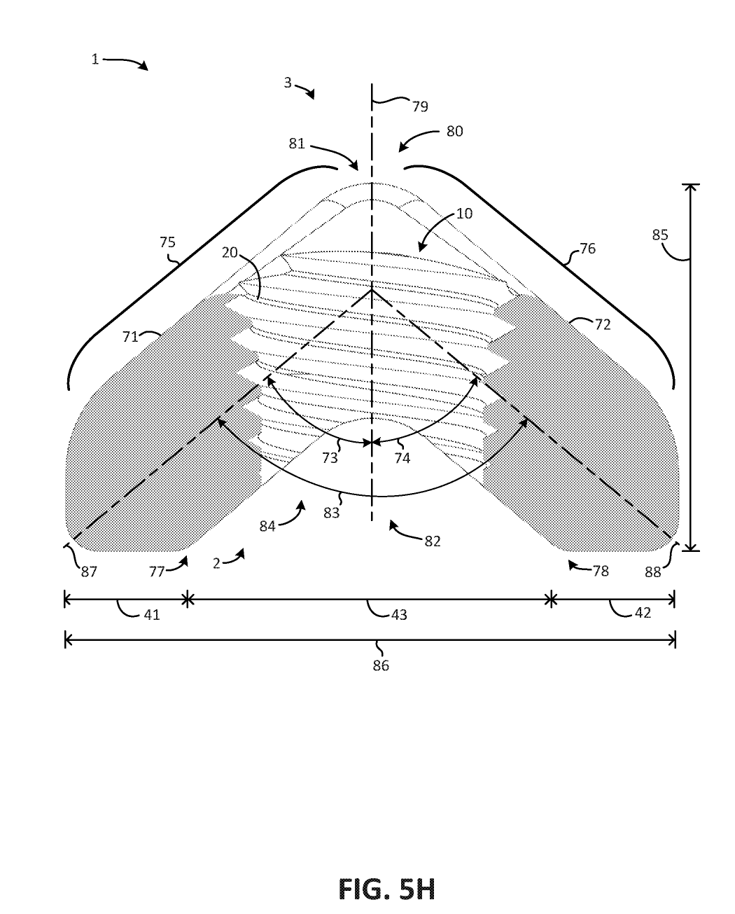

FIGS. 5A-5H show various views of an angled bone plate 1, according to an embodiment of the present disclosure. Specifically, FIG. 5A shows a perspective top view of the angled bone plate 1; FIG. 5B shows a perspective bottom view of the angled bone plate 1; FIG. 5C shows a side view of the angled bone plate 1; FIG. 5D shows a bottom view of the angled bone plate 1; FIG. 5E shows a top view of the angled bone plate 1; FIG. 5F shows a longitudinal cross-sectional side view of the angled bone plate 1; FIG. 5G shows a longitudinal cross-sectional side view of a plate hole 10 of the angled bone plate 1; and FIG. 5H shows a transverse cross-sectional side view of the angled bone plate 1 and a plate hole 10 of angled bone plate 1.

The angled bone plate 1 may generally include a substrate-facing surface, inferior surface, or bone-facing surface 2, a superior surface or reverse surface 3 opposite the bone-facing surface 2, a ridge 80 extending along a longitudinal plate axis 70 of the angled bone plate 1, a first leg 71 projecting away from the ridge 80 along a first leg axis 87 at a first leg angle 73 with respect to a transverse plate axis 79 of the angled bone plate 1, and a second leg 72 projecting away from the ridge 80 along a second leg axis 88 at a second leg angle 74 with respect to the transverse plate axis 79. The first leg 71 may have a first leg width 75 and the second leg 72 may have a second leg width 76.

In some embodiments, the first leg width 75 may be equal to the second leg width 76, which may result in a symmetrical transverse cross-sectional side view for the angled bone plate 1 (e.g., see FIG. 5H).

In some embodiments, the first leg width 75 may be less than the second leg width 76 (or vice versa), which may result in a non-symmetrical transverse cross-sectional side view shape for the angled bone plate 1.

In some embodiments, the first leg width 75 may be greater than the second leg width 76 (or vice versa), which may also result in a non-symmetrical transverse cross-sectional side view shape for the angled bone plate 1.

In some embodiments, the ridge 80 may include a convex superior ridge surface 81 extending along the longitudinal plate axis 70. In some embodiments, the convex superior ridge surface 81 may have a sharp or pointed shape (e.g. a sharp or pointed corner shape, etc.). In some embodiments, the convex superior ridge surface 81 may include a curved or rounded shape, as shown in the non-limiting example of FIGS. 5A-5H.

In some embodiments, the ridge 80 may also include a concave inferior ridge surface 82 extending along the longitudinal plate axis 70. In some embodiments, concave inferior ridge surface 82 may have a sharp or pointed shape (e.g. a sharp or pointed corner shape, etc.). In some embodiments, the concave inferior ridge surface 82 may include a curved or rounded shape, as shown in the non-limiting example of FIGS. 5A-5H.

In some embodiments, a total leg angle 83 for the angled bone plate 1 may be defined as a sum of the first leg angle 73 and the second leg angle 74.

In some embodiments, the first leg angle may be equal to the second leg angle.

In some embodiments, the first leg angle may be less than the second leg angle.

In some embodiments, the first leg angle may be greater than the second leg angle.

In some embodiments, one or more the first leg angle 73, the second leg angle 74, and/or the total leg angle 83 may be less than 90 degrees. For example, one or more of the first leg angle 73, the second leg angle 74, and/or the total leg angle 83 may comprise any angle (or combinations of angles) between zero and 90 degrees. As one non-limiting example, the first leg angle 73 and the second leg angle 74 may each be less than or equal to 60 degrees, but the sum of the first leg angle 73 and the second leg angle 74 may result in a total leg angle 83 that is greater than 60 degrees, etc.

In some embodiments, one or more the first leg angle 73, the second leg angle 74, and/or the total leg angle 83 may be at least 90 degrees. For example, the first leg angle 73 and the second leg angle 74 may each be less than or equal to 90 degrees, but the sum of the first leg angle 73 and the second leg angle 74 may result in a total leg angle 83 that is at least 90 degrees.

In a preferred embodiment, one or more of the first leg angle 73, the second leg angle 74, and/or the total leg angle 83 may comprise an obtuse angle that is greater than 90 degrees and less than 180 degrees. For example, the first leg angle 73 and the second leg angle 74 may each be less than or equal to 90 degrees, but the sum of the first leg angle 73 and the second leg angle 74 may result in a total leg angle 83 that is obtuse (i.e., greater than 90 degrees and less than 180 degrees).

In these embodiments, a total leg angle 83 that is obtuse may help keep a height 85 of the angled bone plate 1 (e.g., see FIG. 5H) less than or equal to a width 86 of the angled bone plate 1. However, it will also be understood that the height 85 of the angled bone plate 1 may be greater than the width 86 of the angled bone plate 1 for any total leg angle 83 (i.e., obtuse angles, right angles, acute angles, etc.).

A total leg angle 83 that is obtuse may be desirable to help avoid excessive prominence of the angled bone plate 1 above the bone 90, while maintaining most of the improved strength against bending and torsional forces compared to a total leg angle of 90 degrees, where a theoretical maximum strength cross-sectional shape and moment of inertia typically occurs. However, the height 85 of the angled bone plate 1 at a total leg angle 83 of 90 degrees may also result in an angled bone plate 1 that is too prominent for a given clinical application. In contrast, a total leg angle 83 that is obtuse may be able to maintain most of its improved strength against bending and torsional forces, while also reducing the height 85 and prominence of the angled bone plate 1, especially in comparison to the weaker cross-sectional shapes and moments of inertia that are associated with typical flat/curved bone plates, as well as bone plates with a total leg angle 83 that is acute.

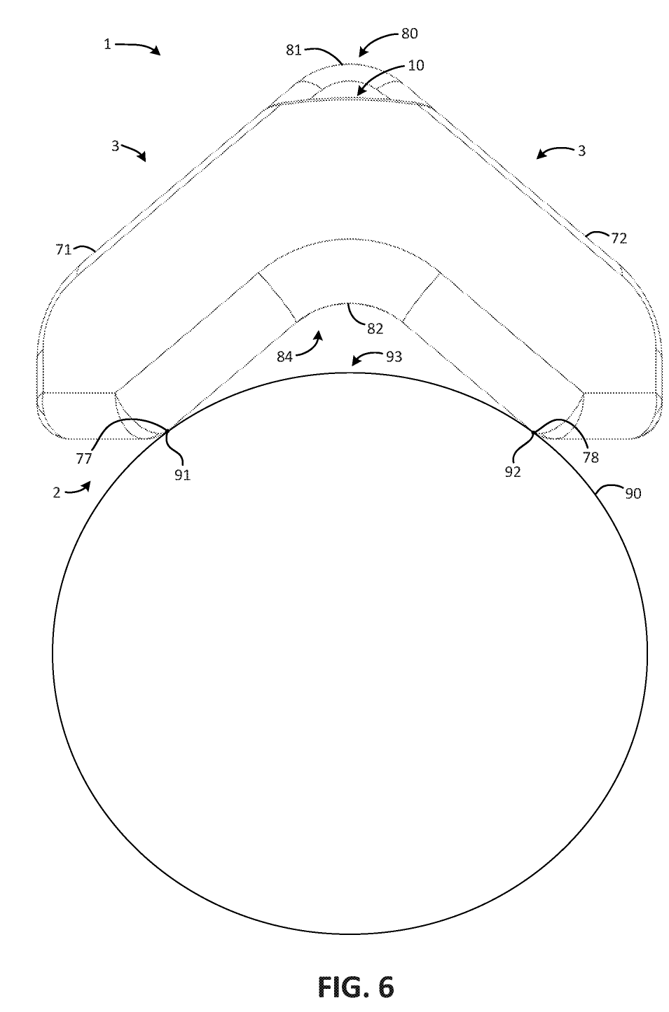

The design of the angled bone plate 1 embodiments described herein can also achieve improved bone coupling characteristics in addition to the improvements in strength previously discussed. For example, the design of the angled bone plate 1 can “straddle” the bone 90 (e.g., see FIG. 6), rather than trying to precisely conform to complex bone surface shapes. In this manner, the angled bone plates of the present disclosure can be easier and quicker to install vs. flat/curved bone plates that may not conform to complex bone surface shapes. Moreover, the angled bone plates of the present disclosure will not unduly restrict blood flow to the periosteum by engaging the bone 90 with via a minimal contact patch to help encourage bone health and healing.

In some embodiments, the first leg 71 may include a first longitudinal contact surface 77 having a first width 41 disposed along at least a portion of the bone-facing surface 2 of the first leg 71, which may be configured to contact the bone 90 along a first longitudinal surface area 91 of the bone 90 (e.g., see FIG. 6). Likewise, the second leg 72 may include a second longitudinal contact surface 78 having a second width 42 disposed along at least a portion of the bone-facing surface 2 of the second leg 72, which may be configured to contact the bone 90 along a second longitudinal surface area 92 of the bone 90. In this configuration, an intermediate portion 84 of the bone-facing surface 2 on the angled bone plate 1 positioned between the first longitudinal contact surface 77 and the second longitudinal contact surface 78 may project away from and/or disengage with an intermediate surface 93 on the bone 90 between the first longitudinal surface area 91 and the second longitudinal surface area 92 of the bone 90. This “straddling” configuration allows quicker and easier installation of the angled bone plate 1 and maintains maximum blood flow to the periosteum (via the minimal contact patch), while improving the overall strength of the angled bone plate 1 as previously described herein.

In some embodiments, the first width 41 of the first longitudinal contact surface 77 may be equal to the second width 42 of the of the second longitudinal contact surface 78.

In some embodiments, the first width 41 of the first longitudinal contact surface 77 may be less than the second width 42 of the of the second longitudinal contact surface 78.

In some embodiments, the first width 41 of the first longitudinal contact surface 77 may be greater than the second width 42 of the of the second longitudinal contact surface 78.

In some embodiments, the intermediate portion 84 of the bone-facing surface 2 may have a third width 43 that may be greater than (or less than, or equal to) a sum of the first width 41 and the second width 42.

In some embodiments, the angled bone plate 1 may comprise or be formed of a rigid or semi-rigid material that may be configured to maintain the total leg angle 83 when the angled bone plate 1 is coupled to the bone 90 via one or more fasteners, as will be discussed below in more detail. However, it will also be understood that some embodiments of the angled bone plate 1 may comprise one or more resilient/bendable materials configured to allow adjustment of the total leg angle 83 of the angled bone plate 1 during the installation process to customize the shape/fit of the angled bone plate 1 with respect to the bone 90.

In some embodiments, the angled bone plate 1 may comprise one or more: metal(s), plastic(s), ceramic(s), polymer(s), composite materials, etc.

In some embodiments, the angled bone plate 1 embodiments described or contemplated herein may include at least one plate hole formed therethrough configured to receive at least one fastener therein to couple the angled bone plate 1 to the bone 90.

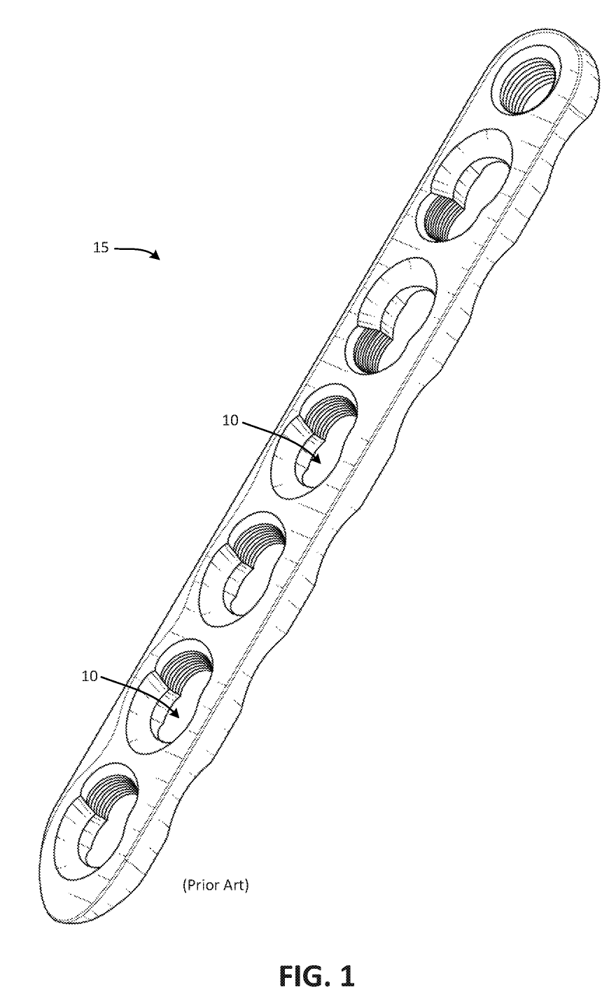

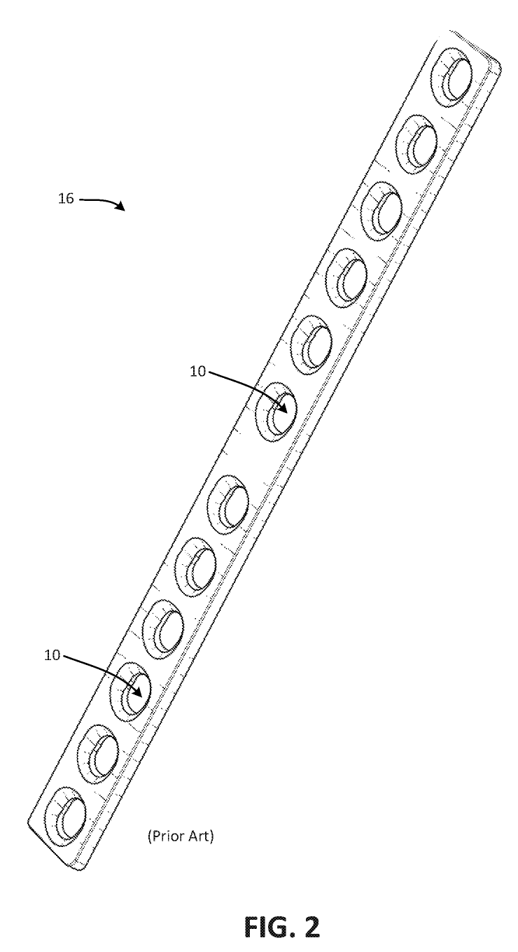

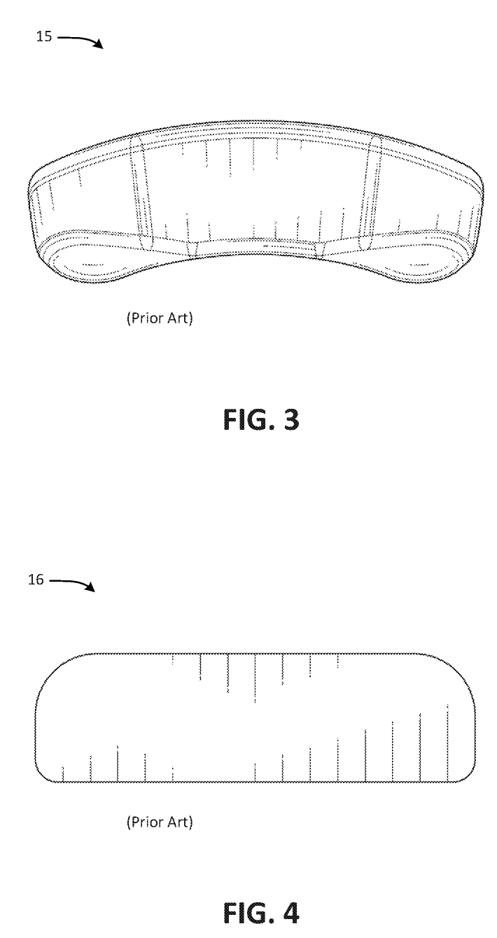

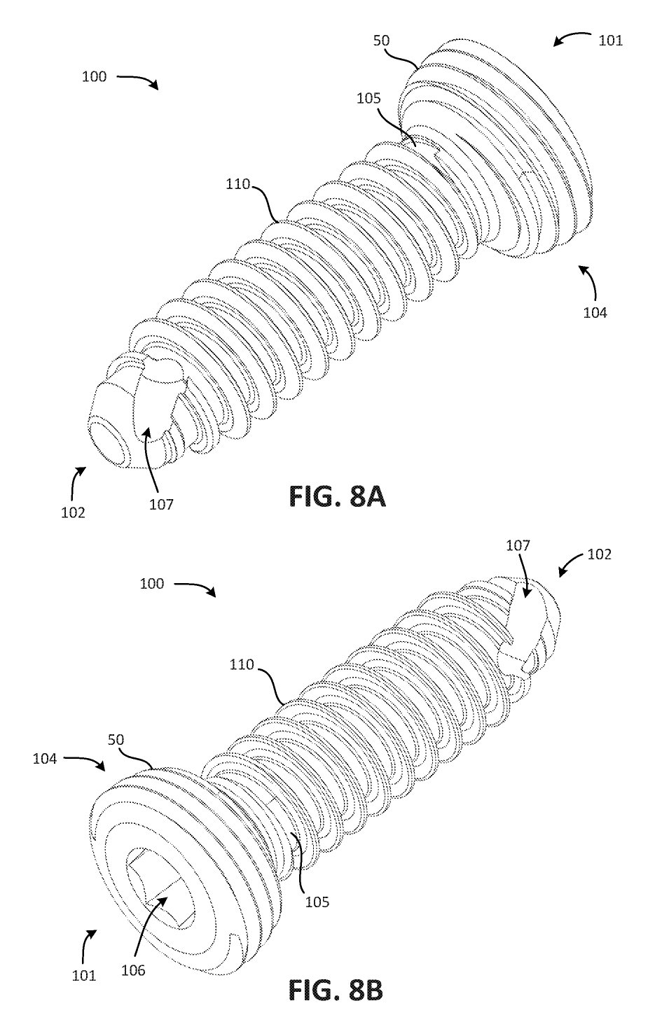

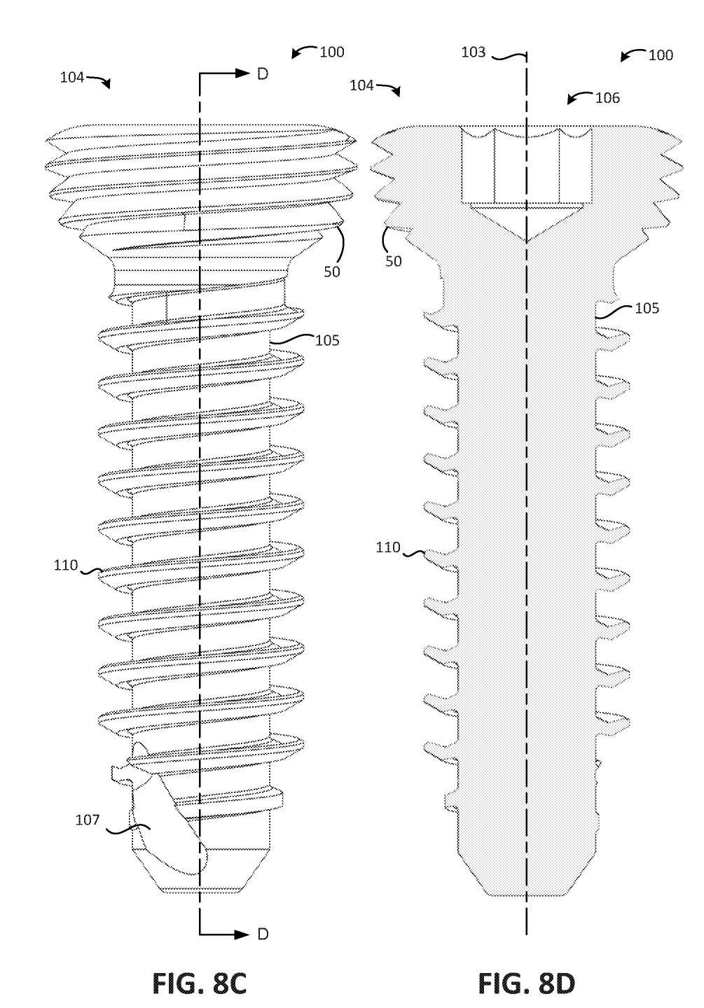

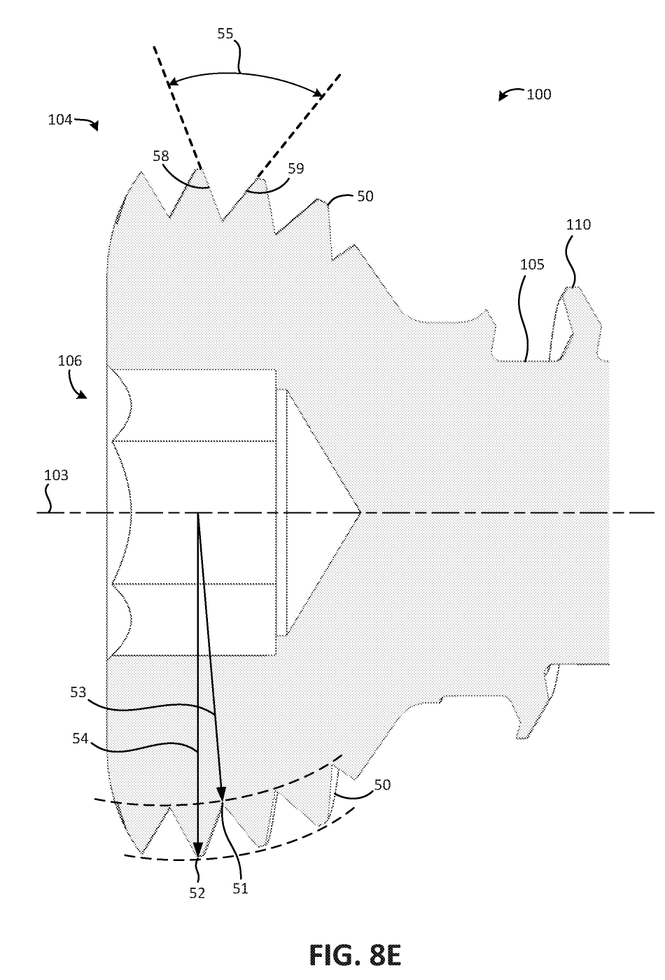

In some embodiments, the plate hole 10 may include at least one of: a fixed-angle locking hole (e.g., see FIGS. 5A-5H), a variable angle locking hole (e.g., that may receive the rounded fastener head shown in FIG. 8E, etc.), a non-locking hole (e.g., see FIG. 2), and a combination hole (e.g., see FIG. 1).

For example, FIG. 5G shows a close up view of a plate hole 10 comprising a fixed-angle locking hole design, according to a non-limiting example of the present disclosure. In this embodiment, the plate hole thread 20 may include a plate hole thread minor diameter that may be configured to taper at a first taper angle 21 with respect to a central axis 5 of the plate hole 10, and a plate hole thread major diameter that may be configured to taper at a second taper angle 22 with respect to the central axis 5 of the plate hole 10.

In some embodiments, the second taper angle 22 of the plate hole thread major diameter may be equal to, or substantially equal to, the first taper angle 21 of the of the plate hole thread minor diameter.

In some embodiments, the second taper angle 22 of the plate hole thread major diameter may be greater than the first taper angle 21 of the of the plate hole thread minor diameter.

In some embodiments, the second taper angle 22 of the plate hole thread major diameter may be less than the first taper angle 21 of the of the plate hole thread minor diameter. For example, in some embodiments the first taper angle 21 of the plate hole thread minor diameter may be about 9.5 degrees or more, and the second taper angle 22 of the plate hole thread major diameter may be about 5.9 degrees or less. However, it will also be understood that, in at least some embodiments, the first taper angle 21 of the plate hole thread minor diameter may be less than 9.5 degrees and/or the second taper angle 22 of the plate hole thread major diameter may be greater than 5.9 degrees.

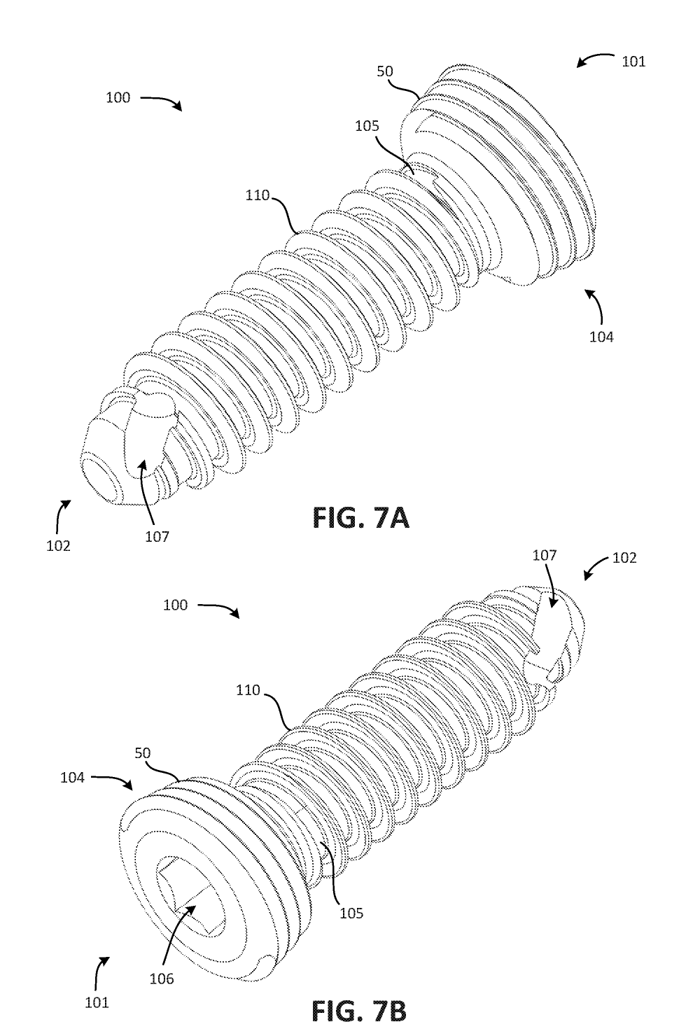

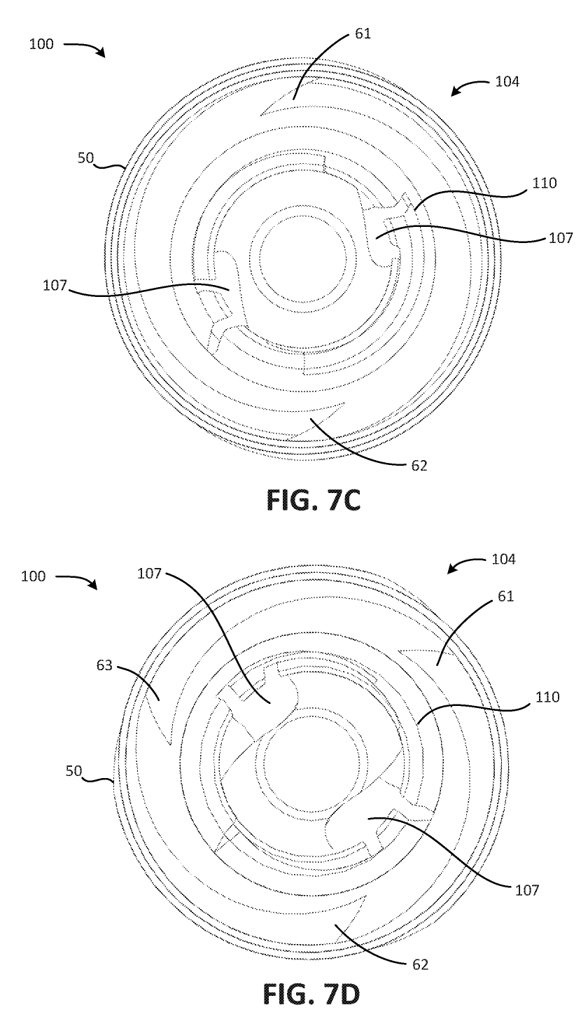

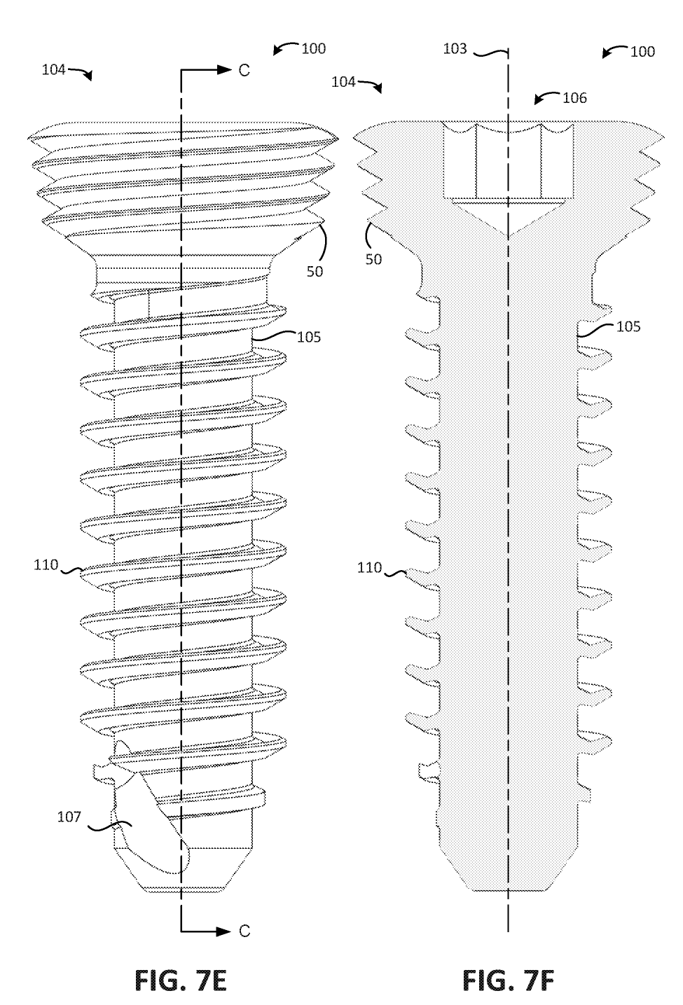

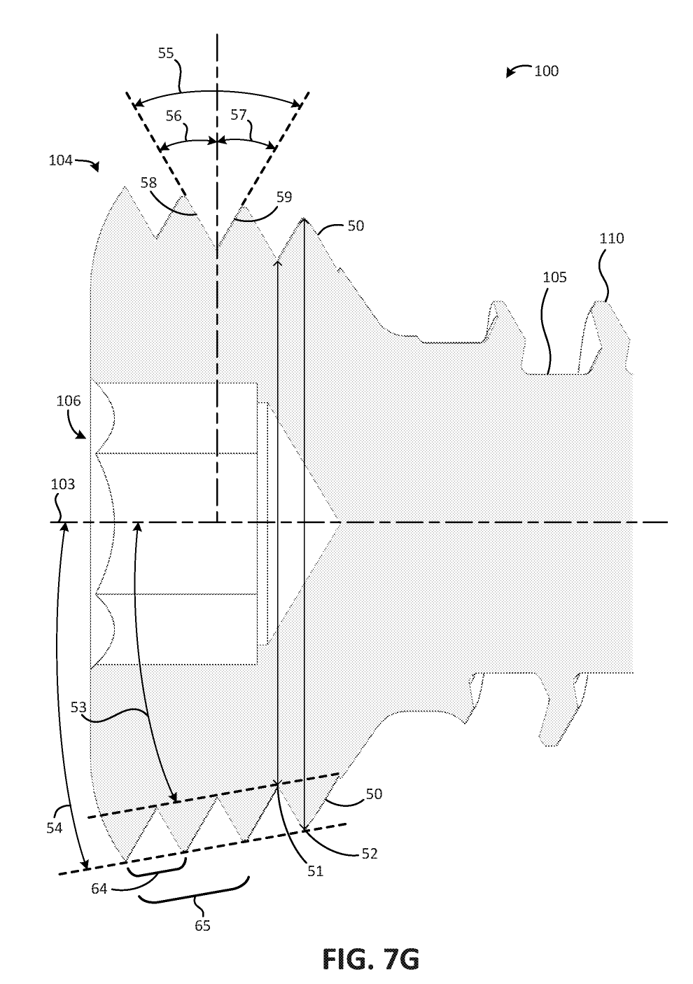

FIGS. 7A-8E show various non-limiting views of example substrate fastener, fastener, or bone fastener embodiments that may be utilized with the angled plates of the present disclosure. Specifically, FIG. 7A is a perspective distal end view of a bone fastener comprising a conically-shaped head; FIG. 7B is a perspective proximal end view of the bone fastener of FIG. 7A; FIG. 7C is a distal end view of the bone fastener with a dual thread start configuration; FIG. 7D is a distal end view of the bone fastener with a triple thread start configuration; FIG. 7E is a side view of the bone fastener of FIG. 7A; FIG. 7F is a cross-sectional side view of the bone fastener taken along the line C-C in FIG. 7E; FIG. 7G is a close up cross-sectional view of the conically-shaped head of the bone fastener; FIG. 8A is a perspective distal end view of another example bone fastener comprising a spherically-shaped head; FIG. 8B is a perspective proximal end view of the bone fastener of FIG. 8A; FIG. 8C is a side view of the bone fastener of FIG. 8A; FIG. 8D is a cross-sectional side view of the bone fastener taken along the line D-D in FIG. 8C; and FIG. 8E is a close up cross-sectional view of the spherically-shaped head of the bone fastener.

In general, the substrate fastener or bone fastener 100 may include a shaft 105 having a proximal end 101, a distal end 102, and a longitudinal axis 103. The bone fastener 100 may also include a head 104 coupled to the proximal end 101 of the shaft 105, a torque connection interface 106 formed in/on the head 104 (in either a male/female configuration), and a self-tapping feature 107 formed in the shaft 105, such as the distal end 102 of the shaft 105, etc.

In some embodiments, the bone fastener 100 may include at least one helical thread or helical thread 110 disposed about the shaft 105. It will be understood that any fastener thread or plate hole thread that is described or contemplated herein may (or may not) utilize any thread configuration, feature, or morphology that is described or contemplated by U.S. Patent Application Serial No. 17/314,028 filed on 05/06/2021, entitled “FASTENING DEVICES, SYSTEMS, AND METHODS” and/or U.S. Patent Application Serial No. 17/877,918 filed on 07/30/2022, entitled “BONE FIXATION DEVICES, SYSTEMS, METHODS, AND INSTRUMENTS”. The above-referenced patent documents are hereby incorporated by reference in their entirety.

In some embodiments, a depth of the helical thread 110 with respect to the shaft 105 may define a major diameter vs. a minor diameter of the shaft 105 alone.

In some embodiments, a major diameter and/or a minor diameter of the bone fastener 100 may be constant or substantially constant along the entire length of the bone fastener 100, along a majority of the length of the bone fastener 100, or along any length of the bone fastener 100.

In some embodiments, the head 104 of the bone fastener 100 may comprise an at least partially conical shape, as shown in FIGS. 7A-7G.

In some embodiments, the head 104 of the bone fastener 100 may comprise an at least partially spherical shape, as shown in FIGS. 8A-8E.

In some embodiments, the bone fastener 100 may include a head locking thread 50 disposed about the head 104 of the bone fastener 100.

In some embodiments, the head locking thread 50 may generally comprise a V-shaped cross-sectional profile, as shown in FIGS. 7G and 8E with respect to the head locking thread superior surface 58 and the head locking thread inferior surface 59. However, it will be understood that the head locking thread 50 may utilize any other cross-sectional shape(s) without departing from the spirit or scope of the present disclosure.

In some embodiments, the head locking thread 50 may comprise a symmetrical thread geometry. For example, the head locking thread 50 may comprise a symmetrical head locking thread angle 55 that may include a superior head locking thread angle portion 56 and an inferior head locking thread angle portion 57.

In some embodiments, the superior head locking thread angle portion 56 may be equal to, or substantially equal to, the inferior head locking thread angle portion 57.

In some embodiments, the non-symmetrical thread geometry of the plate hole thread 20 may be greater than the symmetrical thread geometry of the head locking thread 50.

In some embodiments, the superior head locking thread angle portion 56 may be about 30 degrees, the inferior head locking thread angle portion 57 may be about 30 degrees, and the symmetrical head locking thread angle 55 may be about 60 degrees. However, it will be understood that the superior head locking thread angle portion 56, the inferior head locking thread angle portion 57, and/or the symmetrical head locking thread angle 55 can be any value.

In some embodiments, the head locking thread 50 may include a head locking thread minor diameter 51 and a head locking thread major diameter 52 that may be configured to taper in a proximal-to-distal direction along the head 104 of the bone fastener 100.

In some embodiments, the head locking thread minor diameter 51 may be configured to taper at a third taper angle 53 or spherical radius with respect to the longitudinal axis 103 or center of the head 104, and the head locking thread major diameter 52 may be configured to taper at a fourth taper angle 54 or spherical radius with respect to the longitudinal axis 103 or center of the head 104.

In some embodiments, the third taper angle 53 or spherical radius may be equal to, or substantially equal to, the fourth taper angle 54 or spherical radius.

In some embodiments, the first taper angle 21 of the plate hole thread minor diameter may be less than the fourth taper angle 54 of the head locking thread major diameter 52.

In some embodiments, the first taper angle 21 of the plate hole thread minor diameter may be about 9.5 degrees or less, and the fourth taper angle 54 of the head locking thread major diameter 52 may be about 10 degrees or more. However, it will be understood that, in at least some embodiments, the first taper angle 21 of the plate hole thread minor diameter may be greater than 9.5 degrees, and/or the fourth taper angle 54 of the head locking thread major diameter 52 may be less than 10 degrees.

In some embodiments, the head locking thread 50 may comprise one or more thread starts. For example, the head locking thread 50 may comprise a first head locking thread start 61, a second head locking thread start 62, a third head locking thread start 63, a fourth head locking thread start (not shown), etc.

In some embodiments, the head locking thread 50 may comprise a pitch 64 and a lead 65 which may depend on the number of thread starts utilized by the head locking thread 50.

In some embodiments, at least one of: a pitch of the plate hole thread 20 may be equal to the pitch 64 of the head locking thread 50, a lead of the plate hole thread 20 may be equal to the lead 65 of the head locking thread 50, and a number of thread starts for the plate hole thread 20 may be equal to the number of thread starts for the head locking thread 50.

In some embodiments, the pitch of the plate hole thread 20 may be equal to the pitch 64 of the head locking thread 50, the lead of the plate hole thread 20 may be equal to the lead 65 of the head locking thread 50, and a number of thread starts for the plate hole thread 20 may be equal to the number of thread starts for the head locking thread 50.

In some embodiments, the plate hole 10 may be configured to receive the shaft 105 of the bone fastener 100 therethrough at a fixed angle with respect to the central axis 5 of the plate hole 10 as a fixed-angle/straight locking plate hole. This configuration may provide a strong bone plate hole design in terms of resistance to lateral and back-out forces.

In some embodiments, the plate hole 10 may be configured to receive the shaft 105 of the bone fastener 100 therethrough at any of a plurality of different angles with respect to the central axis 5 of the plate hole 10. In this manner, the bone fastener 100 may exhibit polyaxial adjustability with respect to the angled bone plate 1.

In some embodiments, the plurality of different angles that the shaft 105 of the bone fastener 100 may assume with respect to the central axis 5 of the plate hole 10 may define a conically shaped range of angles between at least +15 degrees to -15 degrees with respect to the central axis 5 of the plate hole 10.

In some embodiments, the plurality of different angles that the shaft 105 of the bone fastener 100 may assume with respect to the central axis 5 of the plate hole 10 may define a conically shaped range of angles between +20 degrees to -20 degrees with respect to the central axis 5 of the plate hole 10. However, it will also be understood that, in at least some embodiments, the plurality of different angles that the shaft 105 of the bone fastener 100 may assume with respect to the central axis 5 of the plate hole 10 may define a conically shaped range of angles between about +90 degrees to -90 degrees or less with respect to the central axis 5 of the plate hole 10.

In some embodiments, the head locking thread 50 may be configured to engage the plate hole thread 20 at a selected angle from among the plurality of different angles and lock the bone fastener 100 to the angled bone plate 1 at the selected angle.

In some embodiments, at least one of the plate hole thread 20 and the head locking thread 50 may be configured to deform and/or cross thread as the head locking thread 50 engages the plate hole thread 20 and locks the bone fastener 100 to the angled bone plate 1 at the selected angle relative to the angled bone plate 1.

Reference throughout this specification to "an embodiment" or "the embodiment" means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Any procedures or methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Recitation of the term "first" with respect to a feature or element does not necessarily imply the existence of a second or additional such feature or element. Elements recited in means-plus-function format are intended to be construed in accordance with 35 U.S.C. §112. It will be apparent to those having skill in the art that changes may be made to the details of the above-described embodiments without departing from the underlying principles set forth herein.

The phrases "connected to", "coupled to", "engaged with", and "in communication with" may refer to any form of interaction between two or more entities, including mechanical, electrical, magnetic, electromagnetic, fluid, and thermal interaction. Two components may be functionally coupled to each other even though they are not in direct contact with each other. The term "coupled" can include components that are coupled to each other via integral formation (e.g., integrally formed as a single piece, etc.), components that are removably and/or non-removably coupled with each other, components that are functionally coupled to each other through one or more intermediary components, etc. The term "abutting" refers to items that may be in direct physical contact with each other, although the items may not necessarily be attached together. The phrase "fluid communication" refers to two or more features that are connected such that a fluid within one feature is able to pass into another feature.

As defined herein the term "substantially" means within +/- 20% of a target value, measurement, or desired characteristic.

The use of "e.g.," and "or" indicates non-exclusive alternatives without limitation, unless otherwise noted. The terms "attached," "coupled," and grammatically related terms refer to the fixed, releasable, or integrated association of two or more elements with or without one or more other elements in between. Thus, the terms "attached" or "coupled" include releasably attaching or fixedly attaching two or more elements in the presence or absence of one or more other elements in between.

As utilized herein with respect to structural features (e.g., to describe shape, size, orientation, direction, relative position, etc.), the terms "approximately," "about," "substantially," and similar terms are meant to cover minor variations in structure that may result from, for example, the manufacturing or assembly process and are intended to have a broad meaning in harmony with the common and accepted usage by those of ordinary skill in the art to which the subject matter of this disclosure pertains. Accordingly, these terms should be interpreted as indicating that insubstantial or inconsequential modifications or alterations of the subject matter described and claimed are considered to be within the scope of the disclosure as recited in the appended claims.

It should be noted that the term "exemplary" and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term "coupled" and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If "coupled" or variations thereof are modified by an additional term (e.g., directly coupled), the generic definition of "coupled" provided above is modified by the plain language meaning of the additional term (e.g., "directly coupled" means the joining of two members without any separate intervening member), resulting in a narrower definition than the generic definition of "coupled" provided above.

References herein to the positions of elements (e.g., "top," "bottom," "above," "below") are merely used to describe the orientation of various elements in the figures. It should be noted that the orientation of various elements may differ according to other exemplary embodiments, and that such variations are intended to be encompassed by the present disclosure.

Although the figures and description may illustrate a specific order of method steps, the order of such steps may differ from what is depicted and described, unless specified differently above. Also, two or more steps may be performed concurrently or with partial concurrence, unless specified differently above.

It is important to note that any element disclosed in one embodiment may be incorporated or utilized with any other embodiment disclosed herein. The devices, systems and methods described herein may be embodied in other specific forms without departing from the characteristics thereof. The foregoing implementations are illustrative rather than limiting of the described systems and methods. The scope of the systems and methods described herein is thus indicated by the appended claims, rather than the foregoing description, and changes that come within the meaning and range of equivalency of the claims are embraced therein.

While specific embodiments and applications of the present disclosure have been illustrated and described, it is to be understood that the scope of the present disclosure is not limited to the precise configurations and components disclosed herein. Various modifications, changes, and variations which will be apparent to those skilled in the art may be made in the arrangement, operation, and details of the devices, systems, and methods disclosed herein.

What is claimed is:

1. An angled bone plate for stabilizing a bone comprising:
a bone-facing surface;
a reverse surface opposite the bone-facing surface;
a ridge extending along a longitudinal plate axis of the angled bone plate;
a first leg projecting away from the ridge at a first leg angle with respect to a transverse plate axis of the angled bone plate, the first leg comprising a first longitudinal contact surface along the bone-facing surface of the first leg that is configured to contact the bone along a first longitudinal surface area on the bone; and
a second leg projecting away from the ridge at a second leg angle with respect to the transverse plate axis of the angled bone plate, the second leg comprising a second longitudinal contact surface along the bone-facing surface of the second leg that is configured to contact the bone along a second longitudinal surface area on the bone;
wherein:
a sum of the first leg angle and the second leg angle comprises a total leg angle of the angled bone plate;
the total leg angle of the angled bone plate is obtuse; and
an intermediate portion of the bone-facing surface of the angled bone plate between the first longitudinal contact surface and the second longitudinal contact surface of the angled bone plate is configured to project away from and disengage an intermediate surface of the bone positioned between the first longitudinal surface area and the second longitudinal surface area on the bone.

2. The angled bone plate of claim 1, wherein:
the first leg comprises a first leg width; and
the second leg comprises a second leg width.

3. The angled bone plate of claim 2, wherein the first leg width is equal to the second leg width.

4. The angled bone plate of claim 2, wherein the first leg width is less than the second leg width.

5. The angled bone plate of claim 2, wherein the first leg width is greater than the second leg width.

6. The angled bone plate of claim 1, wherein a height of the angled bone plate is equal to a width of the angled bone plate.

7. The angled bone plate of claim 1, wherein a height of the angled bone plate is less than a width of the angled bone plate.

8. The angled bone plate of claim 1, wherein a height of the angled bone plate is greater than a width of the angled bone plate.

9. An angled bone plate for stabilizing a bone comprising:
a bone-facing surface;
a reverse surface opposite the bone-facing surface;
a ridge extending along a longitudinal plate axis of the angled bone plate;
a first leg projecting away from the ridge at a first leg angle with respect to a transverse plate axis of the angled bone plate, the first leg comprising a first longitudinal contact surface along the bone-facing surface of the first leg that is configured to contact the bone along a first longitudinal surface area on the bone; and
a second leg projecting away from the ridge at a second leg angle with respect to the transverse plate axis of the angled bone plate, the second leg comprising a second longitudinal contact surface along the bone-facing surface of the second leg that is configured to contact the bone along a second longitudinal surface area on the bone;
wherein:
a sum of the first leg angle and the second leg angle comprises a total leg angle of the angled bone plate;
the total leg angle of the angled bone plate is at least 90 degrees; and
an intermediate portion of the bone-facing surface of the angled bone plate between the first longitudinal contact surface and the second longitudinal contact surface of the angled bone plate is configured to project away from and disengage an intermediate surface of the bone positioned between the first longitudinal surface area and the second longitudinal surface area on the bone.

10. The angled bone plate of claim 9, wherein the first leg angle is equal to the second leg angle.

11. The angled bone plate of claim 9, wherein the first leg angle is less than the second leg angle.

12. The angled bone plate of claim 9, wherein the first leg angle is greater than the second leg angle.

13. The angled bone plate of claim 9, wherein the ridge comprises:
a convex superior ridge surface extending along the longitudinal plate axis; and
a concave inferior ridge surface extending along the longitudinal plate axis opposite the convex superior ridge surface.

14. The angled bone plate of claim 13, wherein:
the convex superior ridge surface comprises at least one of:
a curved convex superior ridge surface shape; and
a pointed convex superior ridge surface shape; and
the concave inferior ridge surface comprises at least one of:
a curved concave inferior ridge surface shape; and
a pointed concave inferior ridge surface shape.

15. An angled bone plate for stabilizing a bone comprising:
a bone-facing surface;
a reverse surface opposite the bone-facing surface;
a ridge extending along a longitudinal plate axis of the angled bone plate;
a first leg projecting away from the ridge at a first leg angle with respect to a transverse plate axis of the angled bone plate, the first leg comprising a first longitudinal contact surface along the bone-facing surface of the first leg that is configured to contact the bone along a first longitudinal surface area on the bone; and
a second leg projecting away from the ridge at a second leg angle with respect to the transverse plate axis of the angled bone plate, the second leg comprising a second longitudinal contact surface along the bone-facing surface of the second leg that is configured to contact the bone along a second longitudinal surface area on the bone;
wherein:
a sum of the first leg angle and the second leg angle comprises a total leg angle of the angled bone plate;
the total leg angle of the angled bone plate is greater than 60 degrees; and
an intermediate portion of the bone-facing surface of the angled bone plate between the first longitudinal contact surface and the second longitudinal contact surface of the angled bone plate is configured to project away from and disengage an intermediate surface of the bone positioned between the first longitudinal surface area and the second longitudinal surface area on the bone.

16. The angled bone plate of claim 15, wherein:
the first longitudinal contact surface of the angled bone plate comprises a first width; and
the second longitudinal contact surface of the angled bone plate comprises a second width.

17. The angled bone plate of claim 16, wherein the first width is equal to the second width.

18. The angled bone plate of claim 16, wherein the first width is less than the second width.

19. The angled bone plate of claim 16, wherein the first width is greater than the second width.

20. The angled bone plate of claim 16, wherein the intermediate portion of the bone-facing surface comprises a third width that is greater than a sum of the first width and the second width.
